SEQUENCE LISTING (12 sequences)

SEQ ID NO: 1 - protein, 20 aa, Artificial Sequence
  qPCR Primer
GATGAAGCTCTTTGAAGTCG

SEQ ID NO: 2 - protein, 20 aa, Artificial Sequence
  qPCR Primer
AGTCAAAGGCATCCATACAG

SEQ ID NO: 3 - protein, 21 aa, Artificial Sequence
  qPCR Primer
CTAGAAGTCCCCAATGCAGTA

SEQ ID NO: 4 - protein, 21 aa, Artificial Sequence
  qPCR Primer
TCTTGCCTCTGACAGTAGCTT

SEQ ID NO: 5 - protein, 20 aa, Artificial Sequence
  qPCR Primer
AGGAAGAAGCCTGTGGAAAT

SEQ ID NO: 6 - protein, 21 aa, Artificial Sequence
  qPCR Primer
GAATTCTCATCCTTCCACTGA

SEQ ID NO: 7 - protein, 20 aa, Artificial Sequence
  qPCR Primer
GTGAAAGAAGGTCGAGTAGA

SEQ ID NO: 8 - protein, 20 aa, Artificial Sequence
  qPCR Primer
GGTATCGTGTATTAGCAGGT

SEQ ID NO: 9 - protein, 20 aa, Artificial Sequence
  qPCR Primer
AGAAGACGGCTATGAGTTCT

SEQ ID NO: 10 - protein, 20 aa, Artificial Sequence
  qPCR Primer
GTACTTCCCCTTGTTCTTGT

SEQ ID NO: 11 - protein, 21 aa, Artificial Sequence
  qPCR Primer
GAAATCCCATCACCATCTTCC

SEQ ID NO: 12 - protein, 21 aa, Artificial Sequence
  qPCR Primer
GAGGCTGTTGTCATACTTCTC

(19) United States
(12) Patent Application Publication
(10) Pub. No.: US 2018/0282735 A1
(43) Pub. Date: Oct. 4, 2018

(54) PHARMACEUTICAL COMPOSITION FOR NEURODEGENERATIVE BRAIN DISEASE AND SCREENING METHOD FOR THE SAME

(71) Applicants: MACROGEN, INC., Seoul (KR); MACROGEN CORP., Rockville, MD (US)

(72) Inventors: Jeong-Sun Seo, Geumcheon-gu (KR); Hoon Ryu, Seongbuk-gu (KR); Jong Yeon Shin, Geumcheon-gu (KR)

(21) Appl. No.: 15/944,193

(22) Filed: Apr. 3, 2018

(30) Foreign Application Priority Data
Apr. 4, 2017 (KR) 10-2017-0043941
Jul. 26, 2017 (KR) 10-2017-0094904

(51) Int. Cl. C12N 15/113; A61P 25/28; G01N 33/68; C12Q 1/6876; C07K 16/40; G01N 33/50

(52) U.S. Cl. CPC C12N 15/1137; A61P 25/28; G01N 33/6896; C12Q 1/6876; C07K 16/40; G01N 2800/28; C12N 2310/122; C12N 2310/531; C12Q 2600/136; G01N 2333/916; G01N 33/5023

(57) ABSTRACT

The present invention provides a therapeutic composition for prevention or treatment of neurodegenerative brain disease caused by traumatic brain injury, comprising a regulator increasing expression or activity of calcineurin, and a screening method for a therapeutic agent for traumatic brain injury and neurodegenerative brain disease mediated by tau protein.

### FIELD

The present invention relates to a kit or a composition for diagnosis or a pharmaceutical composition for prevention or treatment, and a screening method of the therapeutic agent for neurodegenerative brain disease, more specifically, neurodegenerative brain disease caused by traumatic brain injury.

### REFERENCE TO AN ELECTRONIC SEQUENCE LISTING

The contents of the electronic sequence listing (2097_001US_ST25_SequenceListing.bd; Size: 4 kilobytes; and Date of Creation: May 10, 2018) is herein incorporated by reference in its entirety.

### BACKGROUND

Neurodegenerative brain disease causes is a disease that causes degenerative changes in the central nervous system neurons and causes various symptoms such as impaired motor and sensory function, memory, learning, and inhibition of high-order causative functions such as computational inference. Common diseases include Parkinson's disease, Alzheimer's disease, and memory impairment. Neurodegenerative brain disease is characterized by nerve cell death due to rapid or slow progression of necrosis or apoptosis. Therefore, the understanding of the mechanism of nerve cell death must be done for the prevention, control and development of treatment of central nervous system diseases.

Tau protein consists of 4 parts; N-terminal overhang, proline aggregation domain, binding domain and C-terminal (Mandelkow et al., Acta. Neuropathol., 103, 26-35, 1996). It is known that abnormally hyperphosphorylated or modified Tau in nerve cell of central nerve system causes neurodegenerative brain disease such as tauopathy. Among therapeutic agents for neurodegenerative brain disease, however, Tau-targeting agents are not being developed much and therapeutic agents so far usually target kinase proteins. In addition, the precise mechanism how Tau causes the neurodegenerative brain disease is not determined.

An object of the present invention is to provide therapeutic compositions for neurodegenerative brain disease, in particular neurodegenerative brain disease caused by traumatic brain injury, more specifically Tau protein mediating neurodegenerative brain disease caused by traumatic brain injury. For that, the present invention provides a new target of neurodegenerative brain disease therapy by identifying the mechanism of signal transduction pathway mediating phosphorylation, aggregation, and neurotoxicity of tau protein.

In addition, the present invention provides a diagnostic kit or a composition, a pharmaceutical preparation for prevention or treatment of neurodegenerative brain disease, screening methods for the therapeutic agents of neurodegenerative brain disease, especially neurodegenerative brain disease caused by traumatic brain injury, by using the mechanism of Tau-mediating neurodegenerative brain disease caused by traumatic brain injury.

### SUMMARY

The present inventors applied in vitro cell line model and in vivo mouse model as shown in the embodiments to validate how transcriptome changes lead to neuropathological changes in neurodegenerative brain disease caused by traumatic brain injury. Since neurodegenerative brain disease caused by traumatic brain injury and Alzheimer's disease show a similar pathology in terms of increased tau hyperphosphorylation and tauopathy, the present inventors analyzed whether altered transcriptome signatures are associated with the tau phosphorylation pathway in neurodegenerative brain disease caused by traumatic brain injury and Alzheimer's disease. In addition, we verified the immunoreactivity of p-Tau in the postmortem brain tissue of neurodegenerative brain disease caused by traumatic brain injury and Alzheimer's disease.

The term “PP2B”, also known as calmodulin dependent phosphatase, protein phosphatase2B, or PP2B, is one of Serine-Threonine phosphatases regulated by Ca2+/Calmodulin, whose the whole enzyme is made by 1:1 binding of 2 different subunits; A and B.

It is weak on Okadaic acid inhibition and its IC₅₀ is about 10⁻⁸M. The catalytic subunit A has a molecular weight of about 60,000 and a calmodulin binding domain on C-terminal.

Five isoforms are known as α1, α2, β1, β2, β3 which shows 50% of homology with other phosphatase family protein PP1 or PP2A. Regulatory subunit B (Mᵣ=19,000) binds to Ca²⁺. It shows 35% and 29% amino acid homology with calmodulin and troponin C, respectively. It plays important role in signal transduction pathway of immune cells. Cyclosporine A or FK506 as an immunosuppressive drug inhibits specifically enzyme activity by binding to cyclophilin or FKBP respectively.

“Tau” is a microtubule-associated protein and one of IDP (intrinsically disordered proteins) which is unstructured naturally. It interacts with tubulins to stabilize and to stimulate of microtubules. It is previously reported that hyperphosphorylation of tau protein forms NFT (neurofibrillary tangles) and various translocators, and neurodegenerative brain disease is developed by abnormal aggregation (Lee et al., Annu. Rev. Neurosci., 24, 1121-1159, 2001; Bergeron et al., J. Neuropathol. Exp. Neurol., 56, 726-734, 1997; Bugiani et al., J. Neuropathol. Exp. Neurol., 58, 667-677, 1999; Delacourte et al., Ann. Neurol., 43, 193-204, 1998; Ittner and Gotz, Nat. Rev. Neurosci., 12, 65-72, 2011).

In addition, tau protein isolated from microtubules forms misfolded structure which is easy to bind to other tau proteins. They undergo the following aggregation process; β-sheet structure formation, oligomer with several proteins, pre-fibril. (Y. S. Kim, D. J. Kim, O. Hwang, Y. Kim, InTech, 2012, 99-138). Aggregated tau forms neurofibrillary tangles (NFTs), and neurodegenerative brain diseases with these pathological features are collectively referred to as tauopathy. (D. R. Williams, Intern Med J, 2006, 36(10), 652-60). Therefore functional abnormality of tau protein by modification and aggregation is becoming as one of the major causes of Alzheimer's disease and neurodegenerative brain disease. Abnormal aggregation and hyperphosphorylation of tau directly induces degeneration of neuronal cells and it is a common pathogenesis of various degenerative brain disease as well as Alzheimer's disease.

CTE is a progressive neurodegenerative disease that shows clinical symptoms including short-term memory loss, Parkinsonism, and gait and speech disabilities. Increasing evidence shows that a single or repetitive head injury is a risk factor for AD. Importantly, CTE exhibits several neuropathological features of AD, such as tauopathy, neuropil neurites (NNs), and diffuse senile plaques. Indeed, the abnormal levels of hyperphosphorylated tau protein in neurons and astrocytes of CTE are similar or identical to those of neurofibrillary tangles (NFTs) in AD. However, although CTE neuropathologically resembles AD, the mechanism of how brain injury leads to neuropathological sequelae of tauopathy in CTE remains unknown.

In the present study, we analyzed the changes in the transcriptome associated with CTE. Although the pathophysiology of CTE is complex and involves broad changes in gene expression, reduced PPP3CA expression was observed in CTE. PPP3CA is a subunit of calcineurin (PP2B), which dephosphorylates tau, whose dysfunction has been implicated in the generation of both amyloid precursor protein and hyperphosphorylated tau, the two hallmarks of AD pathology. Previous studies have shown that the excitotoxin-induced dysfunction of serine/threonine protein phosphatase activity induces tau phosphorylation in human neurons and that the downregulation of calcineurin (PPP3CA) causes tau hyperphosphorylation in AD patients and Huntington's disease in mouse model brains.

Importantly, experimental systems showed that reduced PPP3CA expression likely leads to tau hyperphosphorylation in CTE, similar to AD. Notably, hyperphosphorylated tau was localized in neurofibrillary tangles and axonal arborizations, areas where the level of PPP3CA was inversely reduced in both CTE and AD cases. Moreover, in vitro experiments using phosphatase inhibitors on BiFC-Tau cells demonstrated the importance of serine/threonine protein phosphatase activity for tau dephosphorylation.

Animal models of TBI have been developed over the last several decades. The weight-drop animal model of TBI mimics a repetitive brain injury in humans and provides several merits: 1) it is simple and easy to apply repetitive-hit consistently, and 2) it represents the pathophysiological features and symptoms of concussion25. Thus, we established a weight-drop animal model of TBI and investigated how PPP3CA knockdown affects repetitive TBI-induced tau phosphorylation in MAPT (P301L) mice. Consistent with the cellular model, PPP3CA knockdown using AAV-shPPP3CA significantly increased the immunoreactivity of p-tau (S202/T205) in the dentate gyrus of P301L mice after exposure to repetitive head injuries. Together, these findings show that alterations in protein phosphatase expression likely contribute to tauopathy in CTE, which suggests that modulating serine/threonine protein phosphatase expression and activity could be a potential therapeutic strategy for preventing progressive tauopathy in CTE and other neurodegenerative diseases.

Using cell lines and animal models, the present inventors also showed that reduced PPP3CA/PP2B phosphatase activity is directly associated with increases in phosphorylated (p)-tau proteins. These findings provide important insights into PP-dependent neurodegeneration and may lead to novel therapeutic approaches to reduce the tauopathy associated with neurodegenerative brain disease caused by traumatic brain injuries.

Therefore, in an embodiment, the present invention is related to pharmaceutical composition for prevention or treatment of neurodegenerative brain disease comprising calcineurin, more specifically a preparation enables stimulating expression or activity of PPP3CA or PP2B phosphatase (pp), screening method for the therapeutic agents, diagnostic kits or compositions of neurodegenerative brain disease and a diagnosis method using the same.

The neurodegenerative brain diseases include, but are not limited to, neurodegenerative brain disease caused by traumatic brain injury, Alzheimer's disease, Parkinson's disease, and Huntington's disease. In detail, the disease according to the present invention may include neurodegenerative brain disease caused by aggregation of tau protein or increased phosphorylation of tau protein such as tauopathy.

In the present invention, the diagnosis is a concept including both the onset and progress of the disease, and the prognosis after the treatment. In the present invention, the screening refers to the selection of patients who are likely to develop neurodegenerative brain disease from patients who are likely to have neurodegenerative brain disease or those who have a risk factor for neurodegenerative brain disease.

As demonstrated in the examples below, the present inventors identified the activity reduction of PPP3CA or PP2B is related to tauopathy in neurodegenerative brain disease caused by traumatic brain injury such as CTE (Example 4), and decreased PPP3CA or PP2B activity induces phosphorylation and aggregation of tau. Furthermore, the present inventors demonstrated that PPP3CA regulates tau phosphorylation directly and that PPP3CA or PP2B regulates tau dephosphorylation in CTE models (See Example 5 and 6). The result shows that stimulation of expression or activity of PPP3CA or PP2B can induce phosphorylation or aggregation of tau protein and can be used as an effective therapeutic agent of prevention or treatment for neurodegenerative brain disease caused by abnormal tau protein.

Particularly, in one embodiment, the present invention provides a pharmaceutical composition for prevention or treatment of a neurodegenerative brain disease, comprising an agent increasing expression or activity of PPP3CA or PP2B.

The agent increasing expression or activity of PPP3CA or PP2B means a substance acts on PPP3CA or PP2B directly or indirectly to improve, induce, stimulate, or increase the expression or activity of PPP3CA or PP3B. The substance means a substance binds directly or indirectly to PPP3CA or PP2B coding genes, mRNA or proteins, to promote expression or activity of PPP3CA or PP2B. The substance comprises single compounds such as organic compounds or inorganic compounds-, peptides, proteins, aptamers, antibodies, nucleic acids, vectors, biopolymers such as carbohydrates and lipids, a complex of natural products and multiple compounds. Preferably, the compound promoting expression or activity of PPP3CA or PP2B is selected from the group comprising compounds, peptides, peptide mimetics, aptamers, antibodies, and natural products.

The mechanism how the preparation promotes expression or activity of PPP3CA or PP2B are not particularly limited. For example, the substance may act as a mechanism to increase gene expression such as transcription, translation, or convert the inactive form to the active form.

For the PPP3CA or PP2B which nucleic acids and protein sequence is known, a person skilled in the art can produce or screen compounds act as accelerators, peptides, peptide mimetics, aptamers, antibodies, and natural compounds using techniques in the art.

The PPP3CA or PP2B expression or activity accelerator of the present invention can be provided as a form of vector enables to express PPP3CA or PP2B in vivo for applying to gene treatment, etc. Accordingly, the present invention relates to pharmaceutical compositions for prevention or treatment of neurodegenerative brain disease comprising nucleic acids or mRNA encoding PPP3CA or PP2B, preferably recombinant vector containing the nucleic acids, as an active ingredient.

Nucleic acid sequences encoding PPP3CA or PP2B can be mutated by substitution, deletion, insertion, or a combination thereof of one or more bases as long as encodes a protein with equivalent activity. The sequence of nucleic acid molecule may be single or double stranded, and it may be a DNA or RNA (mRNA) molecule.

The vectors of the present invention include, but are not limited to, liposomes, plasmid vectors, cosmid vectors, bacteriophage vectors and viral vectors. Examples of preferred viral vectors in the present invention include adenovirus, adeno-associated virus, retrovirus, lentivirus, herpes simplex virus, alpha virus. The recombinant vector of the present invention can include nucleic acids encoding PPPCA or PP2B and regulatory sequences for its transcription or translation. Particularly important regulatory sequences are the sequences which regulate transcription initiation such as promoters, enhancers. It may also contain regulatory sequences consisting of start codon, stop codon, polyadenylation signal, Kozak, enhancer, signal sequences for membrane targeting and secretion, IRES (Internal Ribosome Entry Site). Such regulatory sequences and nucleic acids encoding PPP3CA or PP2B will have to be operably linked.

As used herein, the term “operably linked” refers to the linkage between nucleic acid sequences is functionally related. Any nucleic acid sequence operably linked means any nucleic acid sequence is positioned to have functional relevance with the other. In the present invention, if any transcription regulatory sequence affects transcription of nucleic acid molecule encoding PPP3CA or PP2B, the transcription regulatory sequence and the nucleic acid sequence are said to be operably linked. In the present invention, the treatment comprises repression or prevention of neurodegenerative brain disease or decrease, relief, reverse of symptoms associated with neurodegenerative brain disease and progression inhibition of neurodegenerative brain disease.

The pharmaceutical composition of the present invention can be prepared as a form of pharmaceutical composition for prevention or treatment of neurodegenerative brain disease further comprising suitable carrier, excipient or diluent commonly used in preparation of pharmaceutical compositions. The carrier may comprise non-naturally occurring carrier. Particularly the pharmaceutical composition may be formulated in the form of oral preparations such as powders, granules, tablets, capsules, suspensions, emulsions, syrups and aerosols, external preparations, suppositories and sterilized injection solutions in a conventional manner respectively.

As a specific example of the carrier, excipient, or diluent which may be comprised in the pharmaceutical compositions of the present invention, include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, Water, methylhydroxybenzoate, propylhydroxybenzoate, talc, magnesium stearate, mineral oil. When formulating, it can be prepared using commonly used excipients such as fillers, extenders, binders, wetting agents, disintegrants, surfactants or diluents.

In the pharmaceutical composition in the present invention, the amount of the included agents promoting expression or activity of PPP3CA or PP2B, can include, but are not limited to, based on the total weight of the final composition, from 0.0001 to 50 wt %, more specifically from 0.01 to 20 wt %.

In another aspect, the present invention provides a method for prevention or treatment of neurodegenerative brain disease comprising a step of administrating the pharmaceutical composition at a pharmaceutically effective amount to individual or subject.

As used herein, the term “individuals” or “subject” may include, but are not limited to, mammals including mice, livestock, and humans, or cultured fishes, who have possibility of degenerative brain disease or who already have one.

The route for the administration of the pharmaceutical compositions for prevention or treatment of neurodegenerative brain disease in the present invention can be anyone of administration route commonly used in the art as long as the target tissue can be reached. The administration of the pharmaceutical composition in the present invention include, but are not limited to, intraperitoneal administration, intravenous administration, intramuscular administration, subcutaneous administration, intradermal administration, oral administration, intranasal administration, intrapulmonary administration, rectal administration, depending on the purpose.

The dosage of the pharmaceutical composition of the present invention can be determined by those skilled in the art considering the purpose of use, the toxicity of the disease, the age, weight, gender, medical history of a patient, and the types of active principles. For example, the pharmaceutical composition of the present invention may be administered in an amount of about 0.1 ng/kg to about 100 mg/kg per adult, specifically about 1 ng/kg to about 10 mg/kg, and the administration frequency of the composition of the present invention can be, but not are limited to, once a day or divided into several doses. The dosage does not limit the scope of the present invention in any way.

In further aspect of the present invention, there is a screening method of therapeutic agent of neurodegenerative brain disease, comprising a step which measures whether calcineurin, more specifically PPP3CA or PP2B phosphatase, is expressed or activated or not in the neural tissue-derived cells treated with the candidate substance.

More specifically, the candidate substance shows expression or activity of calcineurin is increased in candidate-treated samples than untreated samples, are selected as a therapeutic agent of neurodegenerative brain disease caused by traumatic brain injury.

The neural tissue-derived cells may be nerve cells or glial cells, as used herein, the term “candidate substance as a therapeutic agent for neurodegenerative brain disease” is a substance predicted as which can cure the neurodegenerative brain disease comprising all potentially treatable substances such as compounds, peptides, peptide mimetics, proteins, aptamers, antibodies, natural products, genes, mRNAs or vectors. The candidate substance can be used without limitation if the substance is predicted to improve directly or indirectly neurodegenerative brain disease.

The measuring step whether expressed or activated may be used by commonly used expression measuring method in the art without limitations, for example, Western blot co-immunoprecipitation assay, ELISA (Enzyme Linked Immunosorbent Assay), RT-PCR, electrophoresis, immunostaining, and FACS (Fluorescence activated cell sorter) can be used.

The neurodegenerative brain disease is preferably neurodegenerative brain disease caused by traumatic brain injury, but is not limited to, for example, it can be a neurodegenerative brain disease by aggregation or phosphorylation increase of tau protein.

In another aspect of the present invention provides a method of diagnosis for neurodegenerative brain disease caused by traumatic brain injury comprising administering an agent measuring calcineurin activity inhibition. The calcineurin is PPP3CA or PP2B and the agent may be antibodies or aptamers bind to PPP3CA or PP2B specifically.

In another aspect of the present invention also provides a diagnostic kit for neurodegenerative brain disease caused by traumatic brain injury, comprising the agent measuring expression or activation of calcineurin.

The measuring agent may be primers or probes for calcineurin genes, antibodies against calcineurin proteins.

In another aspect of the present invention provides a method for providing information for diagnosis of neurodegenerative brain disease caused by traumatic brain injury, comprising a step to measure activity inhibition of PPP3CA or PP2B. The step to measure activity suppression of PPP3CA or PP2B may be carried out by the diagnosis agent or the diagnostic kit for neurodegenerative brain disease and diagnose the onset of neurodegenerative brain disease caused by traumatic brain injury in case of PPP3CA or PP2B activity suppressed.

By administering the therapeutic composition of the present invention to the individual diagnosed by degenerative brain disease, neurodegenerative brain disease caused by traumatic brain injury can be prevented or treated.

The present invention provides a new target for treatment of neurodegenerative brain disease by identifying specific signal transduction mechanism of phosphorylation, aggregation, neurotoxicity of tau protein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1a is a graph for principal component analysis (PCA) of gene expression profiles. RNA samples are prepared from 4 different regions of 6 CTE samples and 7 normal samples respectively. Transcriptome sequences are analyzed by next generation sequencing analyzer. The graph shows a clear distinction in the transcriptomic landscape between CTE and normal subjects

FIG. 1b and FIG. 1c show 12 distinct modules of co-expressed genes by gene co-expression network analysis with expression values of samples. Gene expression of CTE samples is upregulated in blue modules. Gene expression of CTE samples is upregulated in the other modules.

FIG. 1d is the result of gene set enrichment analysis with Kyoto Encyclopedia of Genes and Genomes pathways using genes in 7 modules with a high correlation to CTE among 12 modules. The blue and cyan modules contain the most genes related to neurodegenerative brain disease. In particular, genes related to calcium signaling, Huntington and Alzheimer's are involved.

FIG. 2a shows the result of KEGG pathway analysis 3,064 genes in the blue module. It shows 872 genes upregulated in CTE samples are composed of genes related to the MAPK pathway associated with Alzheimer's disease. 2,192 genes downregulated are mainly associated with neurodegenerative brain disease and 9 genes (CACNA1F, ATP2A2, GRIN2A, PPP3CA, PPP3CB, PPP3R1, CALM1, CALM2, CALM3, and CALML3) are the candidates related to brain damage. Among them, a PPP3CA gene which is calcium dependent phosphatase is shown.

FIG. 2b shows the expression level of PPP3CA, PPP3CB, and PPP3R1 in normal and CTE samples. All three genes are significantly down-regulated.

FIG. 3a shows the expression of PPP3CA and PPP3CB confirmed by qPCR in 11 normal samples and 11 CTE samples not used for sequencing analysis. The mRNA levels of PPP3CA and PPP3CB are reduced in CTE compared to normal subjects.

FIG. 3b shows Western blot result of PPP4CA and PPP3CB protein expression in normal subjects and CTE. Protein levels of PPP3CA and PPP3CB are reduced but levels of p-tau (S199, 5202/T205, and S396) are elevated in CTE because of the reduction of PPP3CA and PPP3CB.

FIG. 3c shows the densitometry analysis (of Western blot) shows that p-tau (S202/T205) is significantly increased while the levels of PPP3CA and PPP3CB are reduced in CTE.

FIG. 3d and FIG. 3e shows tauopathy of CTE in pathological perspective. The densitometry analysis of immunohistochemistry using 3,3-diaminobenzidine (DAB) shows that p-tau is increased in CTE.

FIG. 4a shows the reduction of PPP3CA expression in Alzhemimer's disease samples by RT-PCR. It is performed to confirm whether PPP3CA expression is reduced in Alzheimer's disease because CTE has similar symptoms with Alzheimer's disease and because it is reported that Alzheimer's disease is mediated by tauopathy.

FIG. 4b and FIG. 4c shows expression of PPP3CA and PPP3CB in normal and Alzheimer's disease samples by Western blot. Two proteins are reduced in Alzheimer samples and p-Tau is increased due to the reduction of two proteins. Densitometry analysis with Western blot is shown. PPP3CA and PPP3CB is reduced and p-Tau (S202/T205) is significantly increased in Alzheimer's disease samples.

FIG. 4d and FIG. 4e shows tauopathy of Alzheimer's disease in pathological perspective. The densitometry analysis of immunohistochemistry using 3,3-diaminobenzidine (DAB) shows that p-tau is increased in Alzheimer's disease such as CTE.

FIG. 4f shows reduction of immunoreactivity of PPP3CA and increase of p-Tau with confocal microscope. It shows that PPP3CA is colocalized with p-Tau in neurofibrillary tangles of cerebral cortex of Alzheimer's disease.

From FIG. 5a to FIG. 5e shows that protein phosphatase 2B (PP2B) inhibitors increase tau phosphorylation and tau aggregation in tau-BiFC cell lines. In tau-BiFC system, full-length tau is fused to non-fluorescent N- or C-terminal fragments of Venus fluorescence protein (VN173 or VC155), and both tau constructs were stably expressed in cells. Only when tau assembles, Venus protein could be matured, thereby activating its fluorescence signal. The system were stably expressed in two cell lines (SH-SY5Y and HEK293) and treated PPP3CA inhibitors (Okadaic acid, cyclosporine A & Deltamethrin). Inhibitor-treated cells shows p-Tau and increase of tau accumulation. It shows PPP3CA directly associated with tauopathy.

FIG. 6a and FIG. 6b shows that PPP3CA regulates dephosphorylation of tau protein in cell lines models. GFP-PPP3CA, GFP-GSK3B and tau-GFP is transduced into the cells. GFP-PPP3CA experiment was performed with concentration dependent manner. Western blot is performed extracted proteins from the cells. The result shows a pattern in which reduced p-tau and the GSK3B protein expression depend on the concentration of the PPP3CA treatment. The result demonstrates that PPP3CA regulates phosphorylation of tau protein.

FIG. 6c shows PPP3CA regulates dephosphorylation of tau protein. A construct that catalytic site of PPP3CA is deleted was transduced and performed Western blot with extracted proteins. The result shows phosphorylation of tau has no changes.

FIG. 6d shows PPP3CA regulates dephosphorylation of tau protein. shRNA construct of PPP3CA was transducted and performed Western blot with extracted proteins. As the result, pattern with decreased PPP3CA and increased p-tau are shown. The result demonstrates that PPP3CA regulates phosphorylation of tau protein as shown in FIGS. 6a and 6b.

FIG. 6e shows PPP3CA/PP2B regulates tau phosphorylation in mouse model system. shRNA of PPP3CA is inserted to adeno-associated viral (AAV) vectors and the construct is transfected to the hippocampal regions of tau-transformed mouse (P301L). The mouse was traumatized by weight drop on the head several times. Like the cell model, mice with significantly reduced expression of PPP3CA are obtained from AAV-shPPP3CA mice.

FIG. 6f shows that knock down of PPP3CA increases the level of p-tau (S202/T205) in an animal model of TBI. The level of p-tau (Ser202/Thr205) was increased by AAV-shRNA for PPP3CA in the hippocampal region of mouse brain but not by AAV-shRNA control in response to TBI.

FIG. 6g. shRNA of PPP3CAi inserted to adeno-associated viral (AAV) vectors and the construct is transfected to the hippocampal regions of tau-transformed mouse (P301L. The mouse was traumatized by weight drop on the head several times. As a result, increase of p-tau is demonstrated in the dentate gyrus of hippocampus.

FIG. 6(h). A schematic illustrating that the deregulation of protein phosphatases increases the level of p-tau and p-tau oligomer formation and leads to pathological neurofilament tangle formation in CTE.

FIG. 7 shows expression of cell-type specific genes of neurons, astrocytes and oligodendrocytes to FIG. out whether the traumatic brain injury is cell-type specific. The result shows while neuron-specific genes were mostly down-regulated in CTE, genes related to astrocyte and oligodendrocyte shoed higher expressions.

FIG. 8a shows the correlation between gene significance for CTE status and module membership in the blue module. Gene significance for CTE status and module membership in blue module was highly significantly correlated.

FIG. 8b shows heatmap underneath demonstrated gene expression of the blue module across CTE and normal subjects. Green means negative correlation; Red means positive correlation.

FIG. 8c shows heatmap represents well-known genes involved in tau phosphorylation.

FIG. 9 is a Western blot result showing that PPP3CB reduces the level of p-tau in a dose-dependent manner. The level of phosphorylated tau is inversely correlated with the level of PPP3CB.

FIG. 10 shows repetitive traumatic brain injury increases the immunoreactivity of p-tau in the hippocampal region of Tau transgenic mice. The level of p-tau was increased by AAV-shRNA for PPP3CA in the CA1, CA2, CA3, and dentate gyrus (DG) of mouse brain but by AAV-shRNA control in response to repetitive traumatic brain injury.

### DETAILED DESCRIPTION

The present invention is explained more in detail by examples below. The embodiments are provided for exemplification purposes only, and are not intended to limit the scope of the invention. Materials and methods will be explained below as reference examples.

### REFERENCE EXAMPLE 1

### Human Tissues

Neuropathological processing of control, AD, and CTE human brain samples was performed according to the procedures previously established for the Boston University Alzheimer's Disease Center (BUADC) and Chronic Traumatic Encephalopathy (CTE) Center. Institutional review board approval for ethical permission was obtained through the BUADC and CTE Center. This study was reviewed by the Boston University School of Medicine Institutional Review Board (Protocol H-28974) and was approved as exempt because the study involved only tissue collected from post-mortem individuals, which are not classified as human subjects. Next of kin provided informed consent for participation and brain donation. The study was performed in accordance with the institutional regulatory guidelines and principles of human subject protection in the Declaration of Helsinki. Specific information of the brain tissues are described in Tables 1 (the clinical information of subject) and 2 (AD subject age, gender and Braak stage). In all cases in which AD was diagnosed at autopsy, AD was stated as the cause of death.

TABLE 1

| Samples | individual | Location | Diagnosis | Age/Sex | CTE Stage | TDP-43 |
|---|---|---|---|---|---|---|
| SF_T1 | T1 | Superior Frontal | CTE | 84/M | 4 | 4 |
| AT_T1 | | Anterior Temporal | | | | |
| PV_T1 | | Posterior Visual | | | | |
| SP_T1 | | Superior Parietal | | | | |
| SF_T2 | T2 | Superior Frontal | CTE | 73/M | 4 | 3 |
| AT_T2 | | Anterior Temporal | | | | |
| PV_T2 | | Posterior Visual | | | | |
| SP_T2 | | Superior Parietal | | | | |
| AT_T3 | T3 | Anterior Temporal | CTE | 80/M | 4 | 1 |
| PV_T3 | | Posterior Visual | | | | |
| SP_T3 | | Superior Parietal | | | | |
| SP_T4 | T4 | Superior Parietal | CTE | 73/M | 3 | 1 |
| SF_T5 | T5 | Superior Frontal | CTE | 76/M | 4 | 2 |
| AT_T5 | | Anterior Temporal | | | | |
| SF_T6 | T6 | Superior Frontal | CTE | 93/M | 4 | 4 |
| AT_T6 | | Anterior Temporal | | | | |
| PV_T6 | | Posterior Visual | | | | |
| SP_T6 | | Superior Parietal | | | | |
| SF_C1 | C1 | Superior Frontal | Normal | 88/M | — | — |
| AT_C1 | | Anterior Temporal | | | | |
| SP_C1 | | Superior Parietal | | | | |
| SF_C2 | C2 | Superior Frontal | Normal | 82/M | — | — |
| AT_C2 | | Anterior Temporal | | | | |
| PV_C2 | | Posterior Visual | | | | |
| SP_C2 | | Superior Parietal | | | | |
| SF_C3 | C3 | Superior Frontal | Normal | 74/M | — | — |
| AT_C3 | | Anterior Temporal | | | | |
| PV_C3 | | Posterior Visual | | | | |
| SP_C3 | | Superior Parietal | | | | |
| SF_C4 | C4 | Superior Frontal | Normal | 67/M | — | — |
| AT_C4 | | Anterior Temporal | | | | |
| PV_C4 | | Posterior Visual | | | | |
| SP_C4 | | Superior Parietal | | | | |
| SF_C5 | C5 | Superior Frontal | Normal | 87/F | — | — |
| AT_C5 | | Anterior Temporal | | | | |
| SP_C5 | | Superior Parietal | | | | |
| PV_C5 | | Posterior Visual | | | | |
| AT_C6 | C6 | Anterior Temporal | Normal | 70/M | — | — |
| SF_C7 | C7 | Superior Frontal | Normal | 63/M | — | — |
| AT_C7 | | Anterior Temporal | | | | |
| SP_C7 | | Superior Parietal | | | | |
| PV_C7 | | Posterior Visual | | | | |

TABLE 2

| Number | Case | Age | Sex | Break stage |
|---|---|---|---|---|
| 1 | Normal | 87 | F | I |
| 2 | Normal | 88 | M | I |
| 3 | Normal | 86 | M | II |
| 4 | Normal | 87 | F | II |
| 5 | Normal | 67 | M | I |
| 6 | Normal | 82 | M | I |
| 7 | Normal | 70 | M | I |
| 8 | Normal | 88 | M | I |
| 1 | AD | 79 | F | VI |
| 2 | AD | 70 | M | VI |
| 3 | AD | 80 | F | V |
| 4 | AD | 92 | M | V |
| 5 | AD | 75 | M | V |
| 6 | AD | 83 | M | VI |
| 7 | AD | 79 | F | VI |
| 8 | AD | 89 | M | I V-V |

### REFERENCE EXAMPLE 2

### RNA Sequencing and Analysis

The samples were prepared for sequencing using the Illumina TruSeq RNA sample preparation kit according to the manufacturer's instructions and sequenced on a HiSeq 2000 platform (Illumina, San Diego, Calif., USA). The 101-bp sequenced paired-end reads were mapped to the hg19 reference human genome using the STAR 2-pass method. We used HTSeq to count the reads aligned to each gene based on the Ensembl gene set. We excluded samples that failed in the library preparation or sequence process. We also excluded samples with fewer than 10 million reads sequenced. Overall, 18 CTE subjects and 24 normal subjects were examined. The normalized read counts were applied to PCA or clustering analysis, which was conducted through R and Cluster 3.0 and visualized via Java Treeview (Anders S, Pyl P, Huber W. HTSeq-A Python framework to work with high-throughput sequencing data. Bioinformatics 2015; 31: 166-169; de Hoon M J, Imoto S, Nolan J, Miyano S. Open source clustering software. Bioinformatics 2004; 20: 1453-1454; Saldanha A J. Java Treeview—extensible visualization of microarray data. Bioinformatics 2004; 20: 3246-3248).

### REFERENCE EXAMPLE 3

### Weighted Gene Co-Expression Network Analysis

Co-expression analysis was performed using the weighted gene co-expression network analysis (WGCNA) method via its R package (v3.0.0) (Langfelder P, Horvath S. WGCNA: an R package for weighted correlation network analysis. BMC Bioinformatics 2008; 9: 559). Overall, 24,682 genes were used to construct each network. To construct the networks, the absolute values of Pearson's correlation coefficients were calculated for all possible gene pairs, and the resulting values were transformed such that the final matrix followed an approximate scale-free topology. A connectivity measure (k) per gene was calculated by summing the connection strengths with other genes.

Modules are defined as clusters of densely interconnected genes; by default, the modules are indicated by branches of a hierarchical clustering tree using a dissimilarity measure. Each module is subsequently assigned a color. The gene expression profiles of each module were summarized according to the module eigengene (defined as the first principal component of the module expression levels). A measure of gene significance (GS) was computed to evaluate a gene's correlation with a phenotype. The intra-modular connectivity (k-within) was calculated for each gene by summing the connection strengths with other module genes and dividing this number by the maximum intra-modular connectivity. The adjacency threshold for including edges in the output was set at 0.2. The eigengene-based connectivity of a gene in a module is defined as the correlation of the gene with the corresponding module eigengene.

### REFERENCE EXAMPLE 4

### Enrichment Analyses Based on Kyoto Encyclopedia of Genes and Genomes (KEGG)

Functional annotation analysis was used to assign biological relevance to the gene network modules using Gene Set Enrichment Analysis (GSEA) with KEGG pathway (Subramanian A, Tamayo P, Mootha V K, Mukherjee S, Ebert B L, Gillette M A et al. Gene set enrichment analysis: a knowledge-based approach for interpreting genome-wide expression profiles. Proc Natl Acad Sci USA 2005;102: 15545-15550). The enrichment of the KEGG terms in each module was evaluated based on the hypergeometric test. The output from GSEA is an enrichment score that describes the imbalance in the distribution of ranks of gene expression in each gene set. The number of genes in the overlap (k) was set at ≥3. The enrichment score was normalized according to the size of the gene sets, which were subsequently ranked according to the normalized enrichment score. The default False Positive Rate (FDR) q-value setting (FDR q-value<0.05) was used as the cut-off.

### REFERENCE EXAMPLE 5

### Tau BiFC Cell Lines and Culture

The establishment of the HEK293/tau-BiFC cell line was previously described (Tak H, Hague M M, Kim M J, Lee J H, Baik J H, Kim Y et al. Himolecular fluorescence complementation; lighting-up Tau-Tau interaction in living cells. PLOS One 2013; 8: e81682). Venus fluorescence protein amino-terminal fragment (VN 173) and carboxyl-terminal fragment (VC 155), which are independently non-fluorescent, were fused to full-length tau proteins. For neuronal cell expression, tau-BiFC constructs (pCMV6-hTau40-VN173 and pCMV6-hTau-VC155) were transfected into SH-SY5Y. SH-SY5Y/tau-BiFC stable cells were selected using Geneticin (200 μg/ml). The phosphorylation of tau resulted in the assembly, or aggregation, of two tau molecules, which enabled the maturation of the Venus protein to emit a fluorescence signal. All established cell lines were grown in Dulbecco's modified eagle medium containing 10% fetal bovine serum, 10,000 units/ml penicillin, 10,000 μg/ml streptomycin and 1 μg/ml of Geneticin at 37° C. in a humidified atmosphere containing 5% CO2.

### REFERENCE EXAMPLE 6

### Live Cell Imaging and Analysis

For microscopic image analysis, HEK293 tau-BiFC and SH-SY5Y tau-BiFC cells were plated onto a black 384-well plate. The next day, tau-BiFC cells were treated with okadaic acid (O8010, Sigma), cyclosporine A (C3662, Sigma) or deltamethrin (D9315, Sigma) at various concentrations. After incubation for 29 hours, the plate was imaged using the Operetta® high contents imaging system (PerkinElmer). The cellular intensities of tau-BiFC fluorescence were analyzed using the Harmony® 3.1 analysis software. Each experiment was performed in triplicate. The bar graphs for BiFC intensity indicate the means ±SEM from three independent experiments.

### REFERENCE EXAMPLE 7

### Confocal Microscopy

Tissues and cells were immunostained for p-Tau (S199) (rabbit polyclonal, 1:200; Abcam), p-Tau (S202/T205) (mouse monoclonal, 1:500; Thermo Scientific), and PP2B/PPP3CA (rabbit polyclonal, 1:200; SantaCruz Biotech.) according to a previous report (Rahman A, Grundke-Iqbal I, Iqbal K. PP2B isolated from human brain preferentially dephosphorylates Ser-262 and Ser-396 of the Alzheimer disease abnormally hyperphosphorylated tau. J Neural Transm. 2006; 113: 219-230). For confocal microscopy, the specimens were incubated for 1 hr with fluorescence (FITC)-conjugated secondary antibody (Vector, Burlingame, Calif.) and Cy3-conjugated secondary antibody (Jackson Lab) after the primary antibody incubation. The images were analyzed using a Spinning Disk Confocal microscope (IX2-DSU, Olympus). Preabsorption with excess target protein or omission of primary antibody was used to demonstrate antibody specificity and determine the background generated from the detection assay.

### REFERENCE EXAMPLE 8

### Immunohistochemistry

Immunohistochemistry was performed as previously described (Lee J, Hwang Y J, Shin J Y, Lee W C, Wie J, Kim K Y et al. Epigenetic regulation of cholinergicreceptor M1 (CHRM1) by histone H3K9me3 impairs Ca2+ signaling in Huntington's disease Acta Neuropathol 2013; 125:727-739). Paraffin-embedded tissues were sectioned in a coronal plane at 10 to 20 μm. The tissue sections were rehydrated, blocked with blocking solution (1% H2O2), and incubated with p-Tau (S202/T205) (1:200), PP2B/PPP3CA (1:200; SantaCruz Biotech), p-Tau (S199) (1:200; Abcam), and anti-III tubulin antibody (1:500 dilutions; SIGMA) for 24 hrs. After washing three times, the slides were processed with Vector ABC Kit (Vector Lab). The immunoreactive signals were developed with DAB chromogen (Thermo Fisher Scientific, Meridian, Rockford, USA) and analyzed under a bright field microscope.

### REFERENCE EXAMPLE 9

### Western Blot Analysis

Western blot analysis was performed as previously described (Rahman A, Ting K, Cullen K M, Braidy N, Brew B J, Guillemin G J et al. The excitotoxin quinolinic acid induces tau phosphorylation in human neurons. PLoS One 2009; 4: e6344). For the detection of p-Tau and other proteins, the blots were probed with anti-p-Tau (S199) (1:1000; AbCam), anti-p-Tau (S396) (1:1000), anti-p-Tau (ATB, 5202/T205) (1:1000), and anti-β-actin (1:10000; Sigma Aldrich) antibodies, followed by treatment with the appropriate secondary antibodies conjugated to horseradish peroxidase (Pierce, 170-6515 and 170-6516). Immunoreactivity was detected using an enhanced chemiluminescence (ECL) kit (Thermo Scientific).

### REFERENCE EXAMPLE 10

### Quantitative Real-Time PCR (qPCR)

Total RNA was extracted from the frozen brain tissues using TRIzol reagent (MRC, TR118) as previously described (Lee J, Hwang Y J, Shin J Y, Lee W C, Wie J, Kim K Y et al. Epigenetic regulation of cholinergicreceptor M1 (CHRM1) by histone H3K9me3 impairs Ca2+ signaling in Huntington's disease Acta Neuropathol 2013; 125:727-739.). Fifty nanograms of RNA was used as a template for quantitative RT-PCR amplification, using SYBR Green Real-time PCR Master Mix (Toyobo, QPK-201, Japan). The primers were standardized in the linear range of the cycle prior to the onset of the plateau. The primer sequences are shown in Table 3, and are qPCR primers used for testing the gene expression.

TABLE 3

| | | | Seq |
|---|---|---|---|
| Gene | Direction | Oligonucleotide | ID No |
| PPP3CA | Forward | 5′-GATGAAGCTCTT | 1 |
| (catalytic | | TGAAGTCG-3′ | |
| domain) | Reverse | 5′-AGTCAAAGGCAT | 2 |
| | | CCATACAG-3′ | |
| PPP3CA | Forward | 5′-CTAGAAGTCCCC | 3 |
| (Exon13) | | AATGCAGTA-3′ | |
| | Reverse | 5′-TCTTGCCTCTGA | 4 |
| | | CAGTAGCTT-3′ | |
| PPP3CA | Forward | 5′-AGGAAGAAGCCT | 5 |
| (inhibitory | | GTGGAAAT-3′ | |
| domain) | Reverse | 5′-GAATTCTCATCC | 6 |
| | | TTCCACTGA-3′ | |
| PPP3CB | Forward | 5′-GTGAAAGAAGGT | 7 |
| | | CGAGTAGA-3′ | |
| | Reverse | 5′-GGTATCGTGTAT | 8 |
| | | TAGCAGGT-3′ | |
| PPP1CA | Forward | 5′-AGAAGACGGCTA | 9 |
| (catalytic | | TGAGTTCT-3′ | |
| domain) | Reverse | 5′-GTACTTCCCCTT | 10 |
| | | GTTCTTGT-3′ | |
| GAPDH | Forward | 5′-GAAATCCCATCA | 11 |
| | | CCATCTTCC-3′ | |
| | Reverse | 5′-GAGGCTGTTGTC | 12 |
| | | ATACTTCTC-3′ | |

GAPDH was used as an internal control. Real-time data acquisition was performed using an LightCyler96 Real-Time PCR System (Roche Diagnostics, Germany) under the following cycling conditions: 95° C. for 1 min×1 cycle, and 95° C. for 15 sec, followed by 60° C. for 1 min×45 cycles. The relative gene expression was analyzed using the LightCyler96 software and expressed as Ct the number of cycles needed to generate a fluorescent signal above a predefined threshold.

### REFERENCE EXAMPLE 11

### Construction and Delivery of rAAV Vectors Containing PPP3CA shRNA

For in vivo gene silencing, the validated mouse shRNA sequences for PPP3CA were cloned into the pSicoR vector using HpaI/XhoI sites (Addgene #21907) and subcloned into the pAAV-MCS vector (Stratagene) using MluI/BgIII sites. The high-titer rAAV vectors were produced in HEK293TN cells using a helper virus-free system. Briefly, the rAAV vectors were produced after co-transfecting with equimolar amounts of a rep/cap/helper plasmid. After incubation for 72 hrs, the cells were lysed, treated with benzonase (Sigma #E1014) and further purified using HiTrap heparin columns (GE healthcare #17-0460-01). Amicon ultra-15 centrifugal filter units (Millipore #UFC9100) were used to concentrate up to the final volume. For the delivery of rAAV-PPP3CA shRNA into the brain, the stereotactic microinjection method was used. rAAV-shControl and AAV-PPP3CA shRNA were delivered into the dentate gyrus (AP: −2, ML: 1.5, DV: −1.85) of 4-month-old wild-type mice and Tau transgenic (P301L) mice as previously described.

### REFERENCE EXAMPLE 12

### Animal Model of TBI

All procedures for the animal study were approved by Institutional Animal Care and Use Committee (IACUC) in the Korea Institute of Science and Technology according to international standards and guidelines. Wild type (C57BLJ6) and Tau transgenic (P301L) mice at 4 months of age were used in this study. The mice were fed a standard laboratory diet and water ad libitum in a controlled environment. The mice received three weight drop-induced closed diffuse TBIs at 3-day intervals. We induced closed diffuse TBI by using a weight-drop device (weight 100 g, fall height 75 cm, angle 90 degree) as described previously but with a slight modification (Mychasiuk R. et al., J. Vis. Exp. 2014; 94: e51820, doi:10.3791/51820). The anatomical locus of impact was adjusted to bregma −1 to ±4. All mice were initially anesthetized with 2% avertin (23 μg/g) IP injection before receiving the weight drop-induced TBI. Sham-injured animals were subjected to the same protocol of anesthesia administration, but no mass was ever dropped. After TBI, the mice were placed in the supine position in a clean cage heated using a commercially available heating pad. The mice were then returned to their home cages after normal behavior (e.g., grooming, walking, exploring) was recovered. We examined animal behaviors based on open field and assessed neurohistological changes. The animals were euthanized at 24 hrs after the last impact, and sham-operated animals were euthanized at 24 hrs after the last anesthesia.

### EXAMPLE 1

### Transcriptome Analysis of CTE

The potential contribution of altered gene expression to the pathogenesis of CTE has not been previously studied. We performed RNA sequencing on extracted RNA from four regions of post-mortem brain (anterior temporal (AT), posterior visual (PV), superior frontal (SF), and superior parietal (SP) cortex) from 6 subjects with neuropathologically verified CTE stage III and stage IV and 7 normal controls (Table 1).

Paired-end RNA-Seq was performed using the Illumina HiSeq 2000 platform, and sequencing reads were aligned to the hg19 reference human genome. The average number of reads was ˜71 million (74 million for normal subjects and 76 million for CTE subjects), with approximately 77.4% of the total reads mapping to the human transcriptome (Table 4).

TABLE 4

| | Read | | | Uniquely | # of | |
|---|---|---|---|---|---|---|
| | Length | # of | | mapped reads | Uniquely | Mapped |
| Samples | (bp) | Reads | Bases | (%) | mapped reads | Bases |
| SF_T1 | 2 × 101 | 89,477,966 | 9,037,274,566 | 79.41% | 71,051,654 | 7,176,217,054 |
| AT_T1 | 2 × 101 | 82,974,818 | 8,380,456,618 | 76.06% | 63,112,408 | 6,374,353,208 |
| PV_T1 | 2 × 101 | 72,850,874 | 7,357,938,274 | 74.44% | 54,232,254 | 5,477,457,654 |
| SP_T1 | 2 × 101 | 59,511,698 | 6,010,681,498 | 75.32% | 44,825,706 | 4,527,396,306 |
| SF_T2 | 2 × 101 | 55,326,844 | 5,588,011,244 | 79.39% | 43,921,866 | 4,436,108,466 |
| AT_T2 | 2 × 101 | 76,292,538 | 7,705,546,338 | 79.89% | 60,947,122 | 6,155,659,322 |
| PV_T2 | 2 × 101 | 52,789,162 | 5,331,705,362 | 75.92% | 40,078,716 | 4,047,950,316 |
| SP_T2 | 2 × 101 | 66,284,570 | 6,694,741,570 | 78.17% | 51,815,358 | 5,233,351,158 |
| AT_T3 | 2 × 101 | 88,249,746 | 8,913,224,346 | 82.20% | 72,538,134 | 7,326,351,534 |
| PV_T3 | 2 × 101 | 104,741,092 | 10,578,850,292 | 82.97% | 86,903,176 | 8,777,220,776 |
| SP_T3 | 2 × 101 | 68,224,762 | 6,890,700,962 | 77.06% | 52,574,352 | 5,310,009,552 |
| SP_T4 | 2 × 101 | 91,831,500 | 9,274,981,500 | 86.16% | 79,118,050 | 7,990,923,050 |
| SF_T5 | 2 × 101 | 80,498,582 | 8,130,356,782 | 81.93% | 65,950,646 | 6,661,015,246 |
| AT_T5 | 2 × 101 | 94,223,304 | 9,516,553,704 | 79.44% | 74,851,080 | 7,559,959,080 |
| SF_T6 | 2 × 101 | 77,081,788 | 7,785,260,588 | 78.87% | 60,795,150 | 6,140,310,150 |
| AT_T6 | 2 × 101 | 81,407,012 | 8,222,108,212 | 69.02% | 56,189,194 | 5,675,108,594 |
| PV_T6 | 2 × 101 | 47,711,984 | 4,818,910,384 | 78.47% | 37,440,362 | 3,781,476,562 |
| SP_T6 | 2 × 101 | 79,633,890 | 8,043,022,890 | 71.99% | 57,324,582 | 5,789,782,782 |
| SF_C1 | 2 × 101 | 67,647,636 | 6,832,411,236 | 57.26% | 38,737,874 | 3,912,525,274 |
| AT_C1 | 2 × 101 | 74,646,302 | 7,539,276,502 | 91.36% | 68,196,390 | 6,887,835,390 |
| SP_C1 | 2 × 101 | 70,227,800 | 7,093,007,800 | 77.35% | 54,317,972 | 5,486,115,172 |
| SF_C2 | 2 × 101 | 76,371,856 | 7,713,557,456 | 60.67% | 46,337,958 | 4,680,133,758 |
| AT_C2 | 2 × 101 | 85,536,650 | 8,639,201,650 | 75.36% | 64,461,774 | 6,510,639,174 |
| PV_C2 | 2 × 101 | 84,707,524 | 8,555,459,924 | 65.46% | 55,447,140 | 5,600,161,140 |
| SP_C2 | 2 × 101 | 87,699,934 | 8,857,693,334 | 77.70% | 68,139,492 | 6,882,088,692 |
| SF_C3 | 2 × 101 | 86,123,180 | 8,698,441,180 | 75.58% | 65,091,718 | 6,574,263,518 |
| AT_C3 | 2 × 101 | 99,450,720 | 10,044,522,720 | 81.71% | 81,264,282 | 8,207,692,482 |
| PV_C3 | 2 × 101 | 90,908,222 | 9,181,730,422 | 70.12% | 63,744,808 | 6,438,225,608 |
| SP_C3 | 2 × 101 | 112,046,048 | 11,316,650,848 | 62.38% | 69,894,916 | 7,059,386,516 |
| SF_C4 | 2 × 101 | 69,153,502 | 6,984,503,702 | 81.46% | 56,334,952 | 5,689,830,152 |
| AT_C4 | 2 × 101 | 86,533,640 | 8,739,897,640 | 79.55% | 68,837,332 | 6,952,570,532 |
| PV_C4 | 2 × 101 | 64,090,504 | 6,473,140,904 | 76.49% | 49,020,506 | 4,951,071,106 |
| SP_C4 | 2 × 101 | 49,689,838 | 5,018,673,638 | 57.41% | 28,525,606 | 2,881,086,206 |
| SF_C5 | 2 × 101 | 53,770,430 | 5,430,813,430 | 87.23% | 46,905,748 | 4,737,480,548 |
| AT_C5 | 2 × 101 | 61,453,168 | 6,206,769,968 | 88.65% | 54,475,924 | 5,502,068,324 |
| SP_C5 | 2 × 101 | 67,454,288 | 6,812,883,088 | 78.49% | 52,947,456 | 5,347,693,056 |
| PV_C5 | 2 × 101 | 81,778,604 | 8,259,639,004 | 88.67% | 72,509,850 | 7,323,494,850 |
| AT_C6 | 2 × 101 | 62,723,588 | 6,335,082,388 | 83.23% | 52,201,812 | 5,272,383,012 |
| SF_C7 | 2 × 101 | 74,618,218 | 7,536,440,018 | 80.35% | 59,953,846 | 6,055,338,446 |
| AT_C7 | 2 × 101 | 43,290,416 | 4,372,332,016 | 86.18% | 37,309,556 | 3,768,265,156 |
| SP_C7 | 2 × 101 | 62,895,188 | 6,352,413,988 | 90.56% | 56,955,946 | 5,752,550,546 |
| PV_C7 | 2 × 101 | 67,292,522 | 6,796,544,722 | 90.14% | 60,654,426 | 6,126,097,026 |
| AT_AD1 | 2 × 101 | 73,765,170 | 7,450,282,170 | 78.71% | 58,061,442 | 5,864,205,642 |
| AT_AD2 | 2 × 101 | 83,992,030 | 8,483,195,030 | 81.82% | 68,722,096 | 6,940,931,696 |
| AT_AD3 | 2 × 101 | 51,885,624 | 5,240,448,024 | 79.58% | 41,288,224 | 4,170,110,624 |
| AT_AD4 | 2 × 101 | 46,649,484 | 4,711,597,884 | 65.74% | 30,667,548 | 3,097,422,348 |

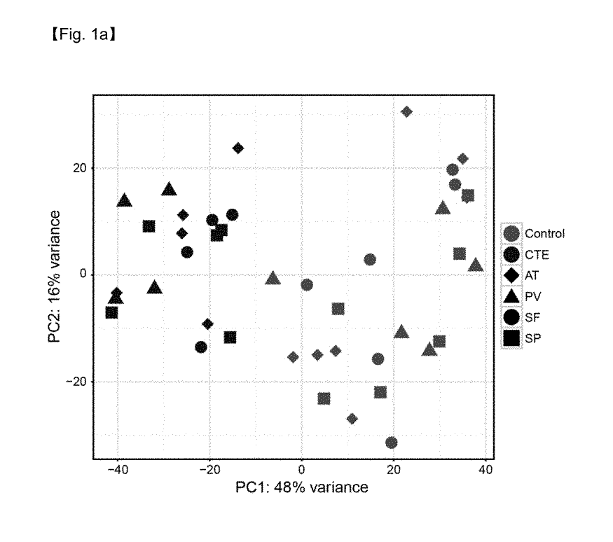

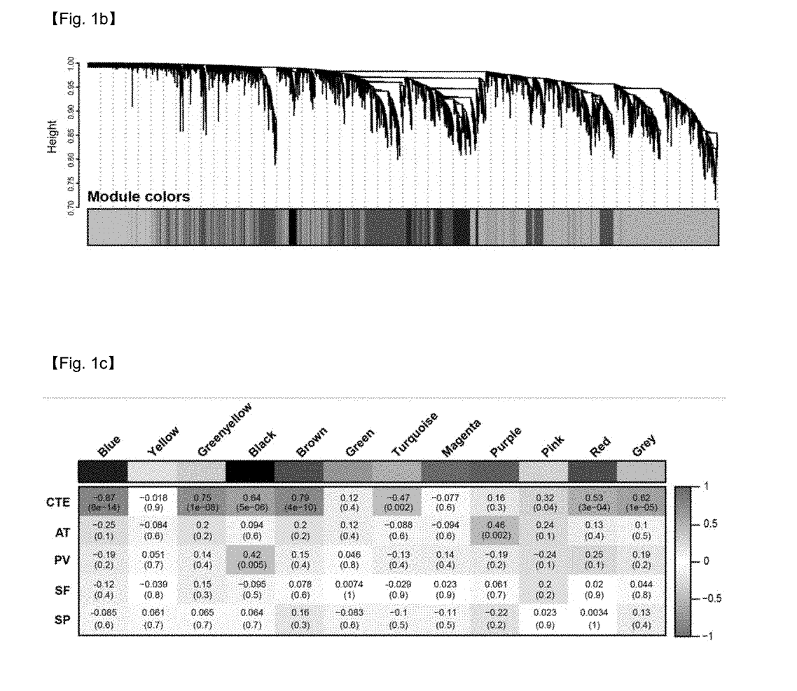

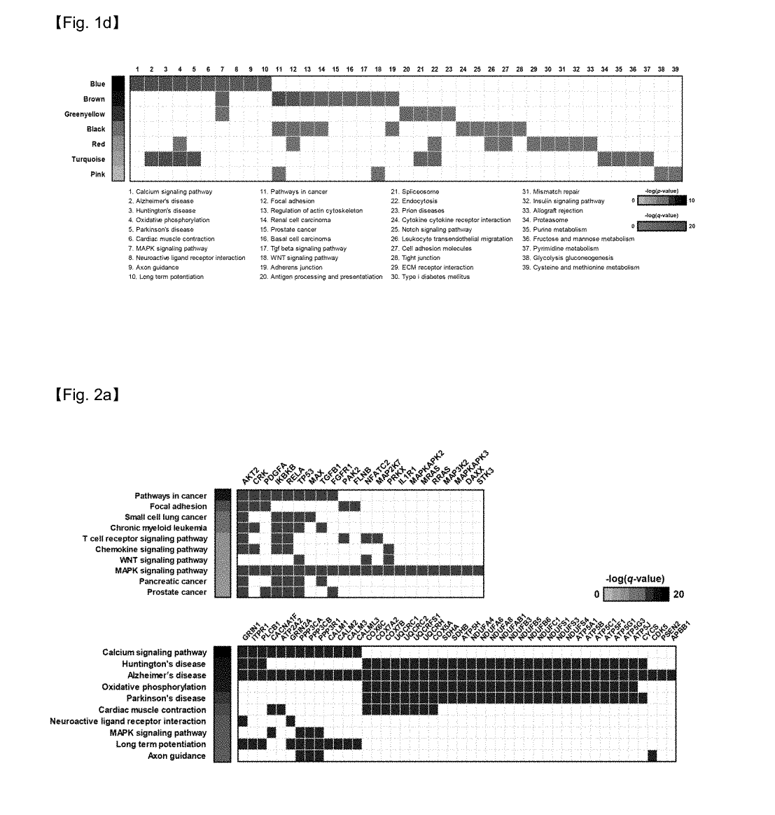

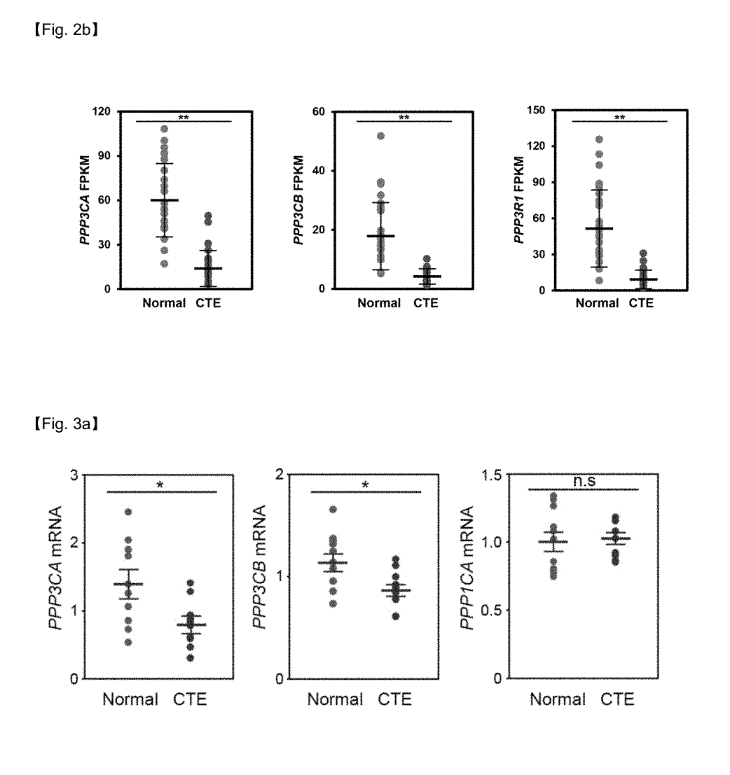

To assess the similarities and differences among samples, we conducted a principal component analysis (PCA) for the RNA-seq samples. The results demonstrated a clear distinction in the transcriptomic landscape between CTE and normal subjects (FIG. 1a).

### EXAMPLE 2

### Gene Network Analysis Identifies Co-Expressed Gene Modules

The inventors applied a weighted gene co-expression network analysis (WGCNA) (Langfelder P. et al. BMC Bioinformatics 2008; 9: 559) to identify CTE associated co-expression modules and their key constituents. WGCNA clusters genes with similar expression patterns in an unbiased manner, thus enabling the biological interpretation of transcriptional patterns. We identified 12 distinct co-expression modules, which varied in size from 74 to 3,355 genes. A total of 13,331 genes did not share similar co-expression with the other genes in the network and were thus not included in any of the modules (FIG. 1b).

The module eigengene represented the expression of all genes classified into that module. When each eigengene expression was correlated against CTE status or other experimental variables, such as brain region (AT, PV, SF, and SP) using Pearson's method, only CTE status significantly correlated positively or negatively with a module eigengenes (FIG. 1c). The blue module (r=−0.87, P=8×10−14) was negatively correlated with CTE subjects, indicating that most of genes in this module were downregulated in the CTE brain, whereas other significant modules for CTE were positively correlated. The results regarding the changing expression patterns of cell type-specific genes suggest that the genes related to both neurons and oligodendrocytes are affected by CTE progression. Specifically, neuron-associated genes were enriched in the blue module and showed decreased expression with CTE (FIG. 7). Consistent with the PCA analysis, the WGCNA analysis revealed no significant differences in the four brain regions. Only AT and PV were positively correlated with purple and black modules, respectively.

To understand the biological relevance of the identified modules highly correlated to CTE, we performed gene set enrichment analysis (GSEA) with Kyoto Encyclopedia of Genes and Genomes (KEGG) pathways (FIG. 1d). Two (blue and turquoise modules) of the seven modules were highly enriched for specific functional pathways that are directly associated with neurodegenerative diseases and neurobiological functions. In particular, the blue module was enriched for functional categories explicitly related to the calcium-signaling pathway, Alzheimer's disease, Huntington's disease and Parkinson's disease and to genes belonging to the MAPK-signaling pathway. The brown and black modules were enriched for genes belonging to pathways involved in cancer, focal adhesion and actin cytoskeleton regulation. This analysis suggests that the large coordinated changes in gene expression are specifically associated with CTE brain.

### EXAMPLE 3

### The Blue Gene Co-Expression Module is Significantly Associated With CTE

The blue module contains 3,064 genes with a high correlation between the members of its module and gene significance for CTE (r=0.85 and P<1×10−200, FIG. 8a). Unsupervised hierarchical clustering performed on gene expression levels of the blue module showed the distinct separation of CTE from normal subjects (FIG. 8b). Eight hundred and seventy-two (872) upregulated and 2,192 downregulated genes were abnormally expressed in CTE. Functional annotation analyses based on KEGG pathways using upregulated and downregulated genes in the blue module are provided in FIG. 2a. The upregulated genes were primarily involved in the MAPK pathway, which is associated with Alzheimer's disease pathogenesis. However, the tau phosphorylation kinase, p38 MAPK (MAPK11, MAPK12, MAPK13, and MAPK14), was not activated or associated with the blue module. Moreover, the expression levels of the other tau phosphorylation kinases, GSK3 (GSK3A and GSK3B), CDKS, and FYN, were not also increased in CTE (FIG. 8c). The downregulated genes were associated with several neurodegenerative diseases. Among these genes, we focused on genes not associated with Huntington's disease and Parkinson's disease, which do not have tauopathy for main disease progression. Using this approach, we identified nine genes that might affect CTE progression: CACNA1F, ATP2A2, GRIN2A, PPP3CA, PPP3CB, PPP3R1, CALM1, CALM2, CALM3, and CALML3. From the abovementioned genes, we identified PPP3CA, encoding a calcium-dependent, calmodulin-stimulated protein phosphatase, which was reported to be decreased in the medial temporal gyrus from AD patient brains (Chiocco M J et al. Fine mapping of Calcineurin (PPP3CA) gene reveals novel alternative splicing patterns, association of 5′UTR trinucleotide repeat with addiction vulnerability, and differential isoform expression in Alzheimer's disease. Subst Use Misuse 2010; 45:1809-1826). In addition, PPP3CB and PPP3R1, which are subunits of PP2B, were significantly decreased in CTE subjects (FIG. 2b).

### EXAMPLE 4

### Reduction of PPP3CA/PP2A activity is Associated With Tauopathy in CTE

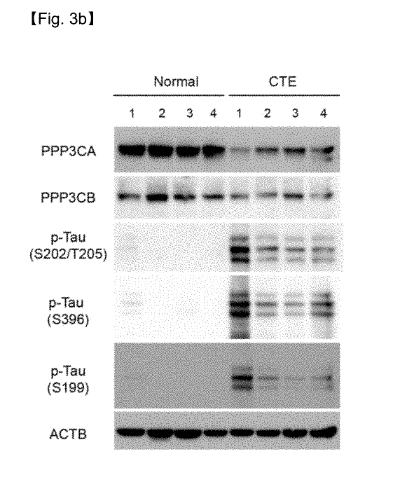

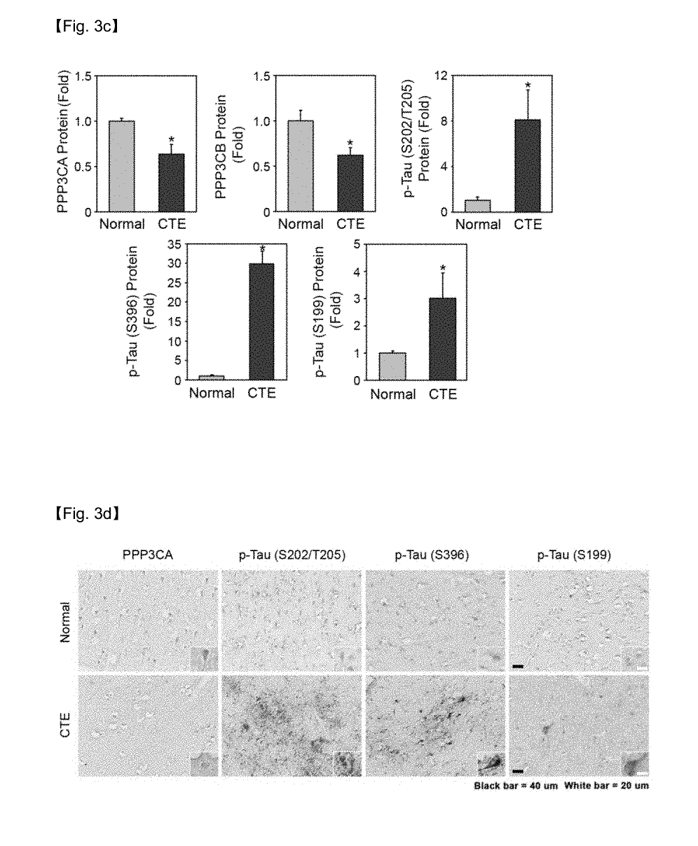

We explored whether altered PPP3CA gene expression also contributes to pathological tau phosphorylation and tauopathy in CTE. First, the downregulation of PPP3CA was confirmed through qPCR in the cortex of human CTE and normal subjects (FIG. 3a). Western blot and densitometry analyses subsequently showed that the levels of PPP3CA were markedly decreased in CTE compared to normal subjects (FIGS. 3b and 3c). Furthermore, the levels of p-tau at S199, S202/T205 and S396, which are the most common sites of tau phosphorylation in neurodegenerative disorders, were increased 3- and 30-fold in CTE. Importantly, the levels of PPP3CA protein and p-tau (S202/T205) in the frontal cortex of CTE patients were inversely correlated. To further investigate the pathological characteristics of tauopathy in CTE, we performed 3,3′-diaminobenzidine (DAB) immunohistochemical staining in the cortex of normal and CTE brains (FIGS. 3d and 3e). Consistent with the qPCR and western blot data, the immunoreactivity of PPP3CA was decreased, and tau was hyperphosphorylated (S199, S396, S205/T208) in CTE.

### EXAMPLE 5

### Reduction of PPP3CA/PP2B Activity is Associated With Tauopathy Also in AD

Because the characteristics of CTE closely overlap with AD both clinically and pathologically, we examined whether PP2B activity is likely decreased in AD, which exhibits the most prevalent tauopathy.

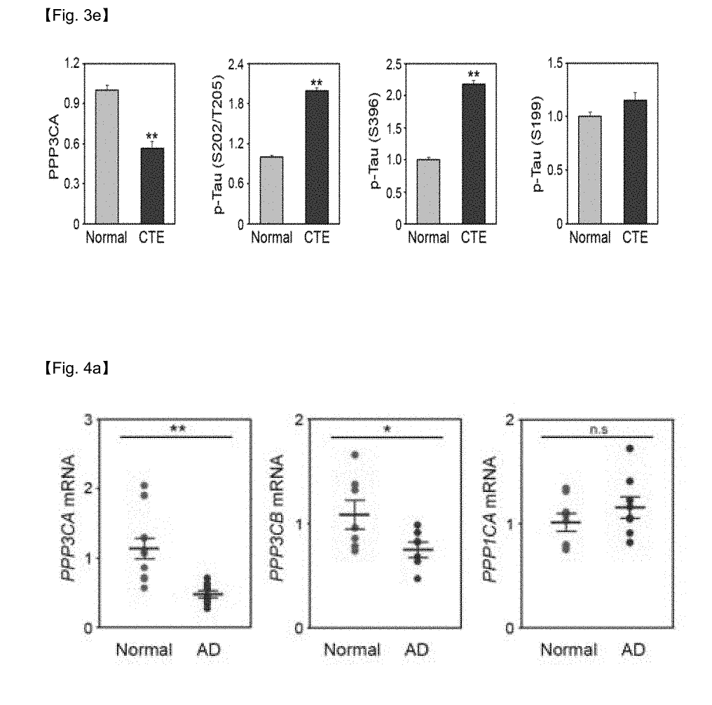

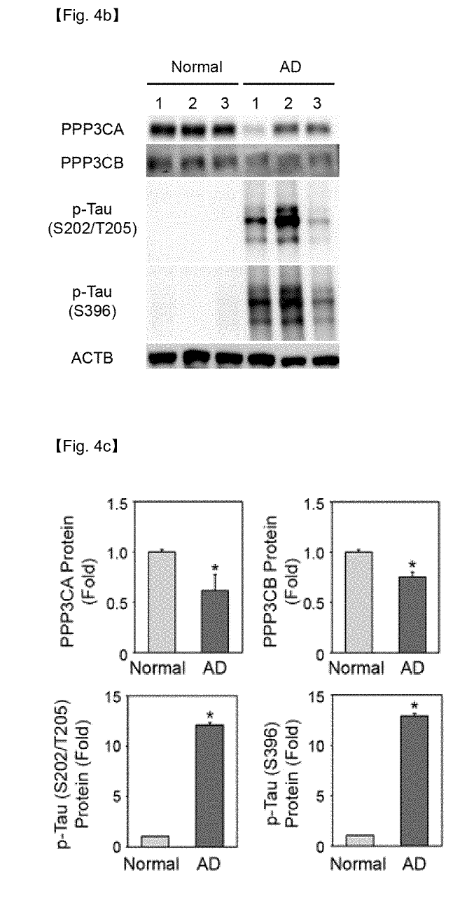

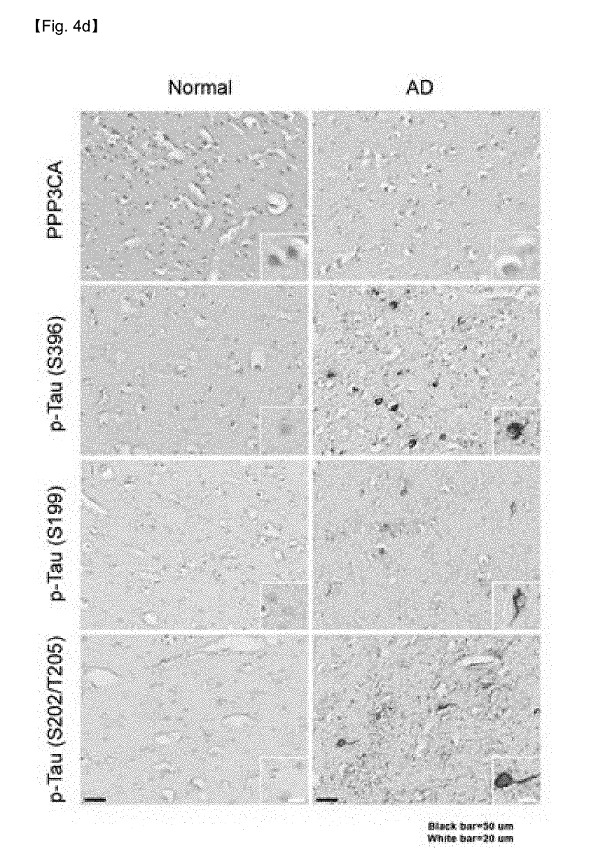

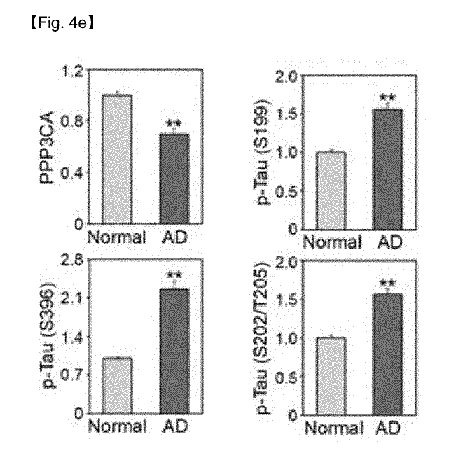

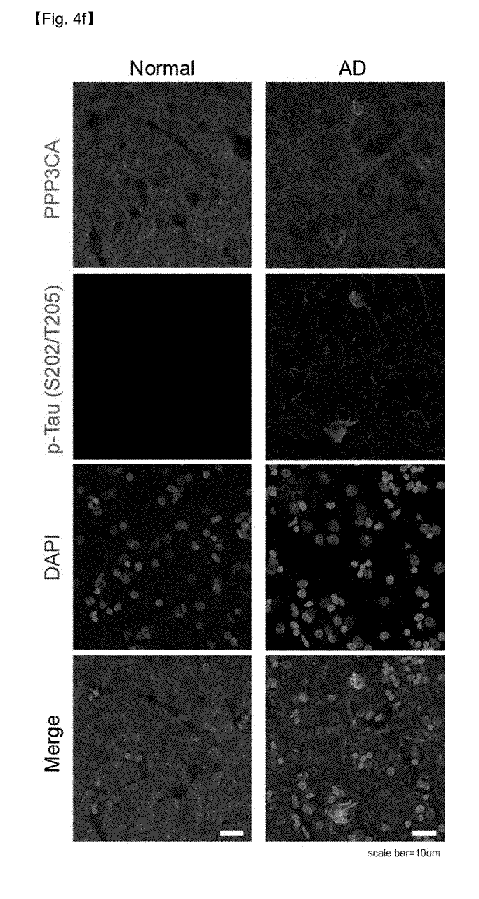

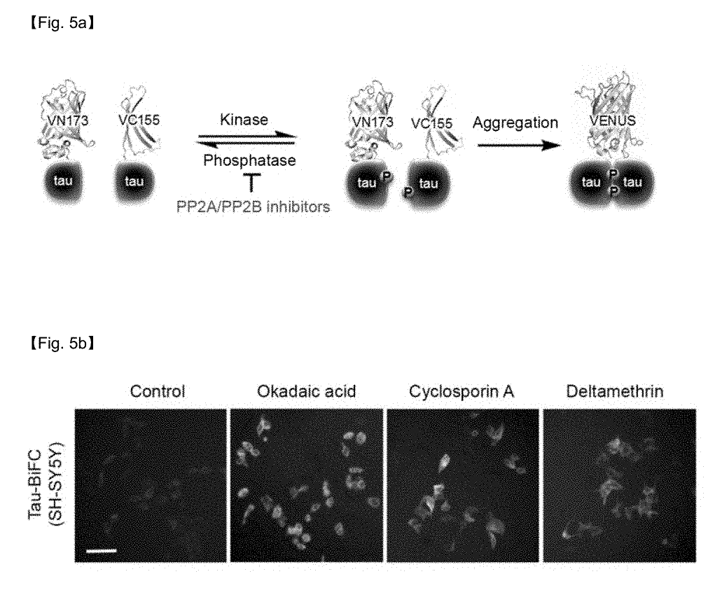

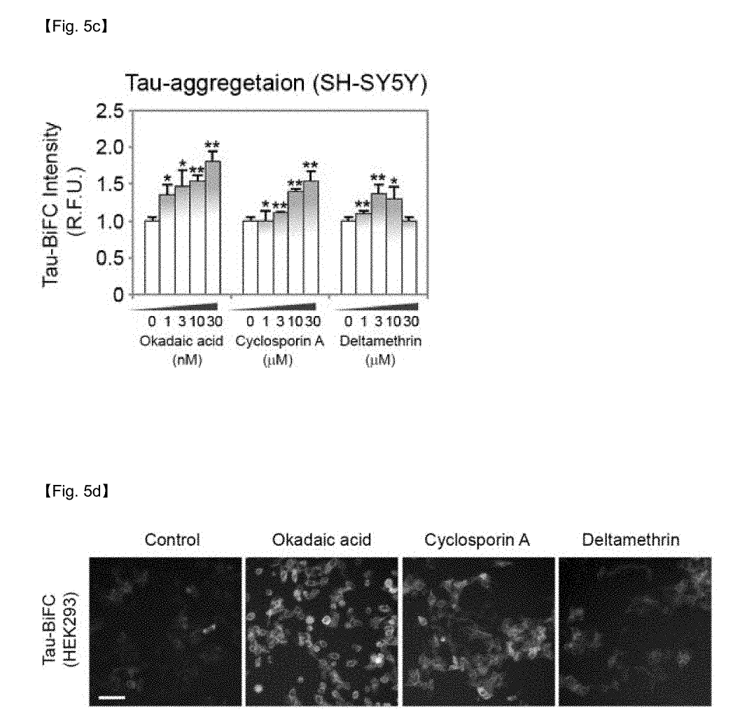

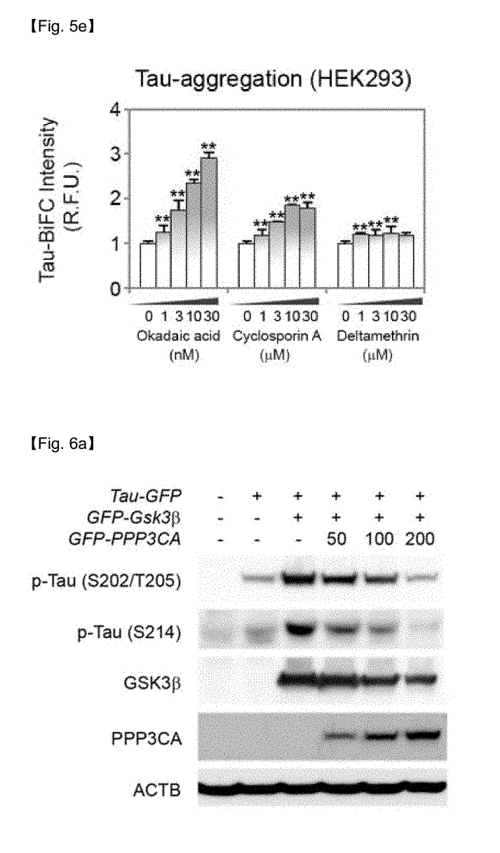

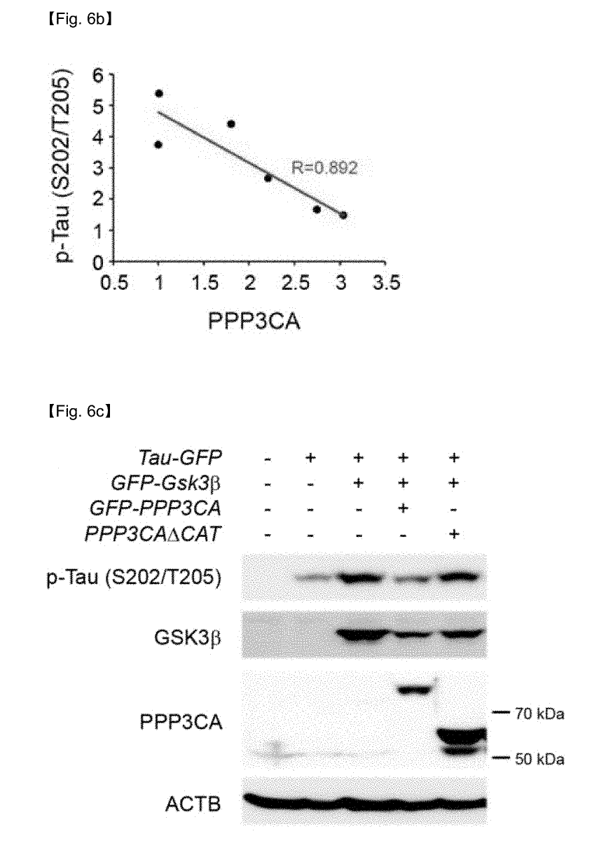

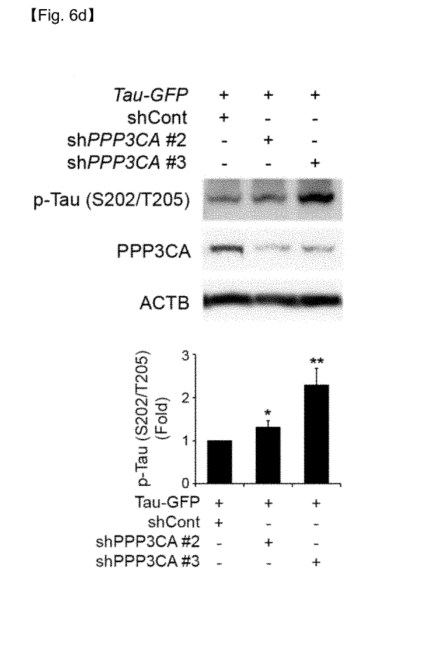

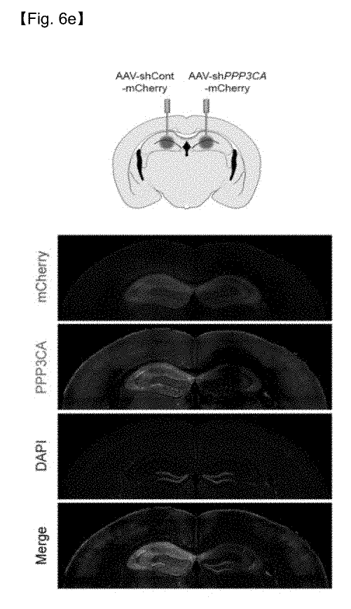

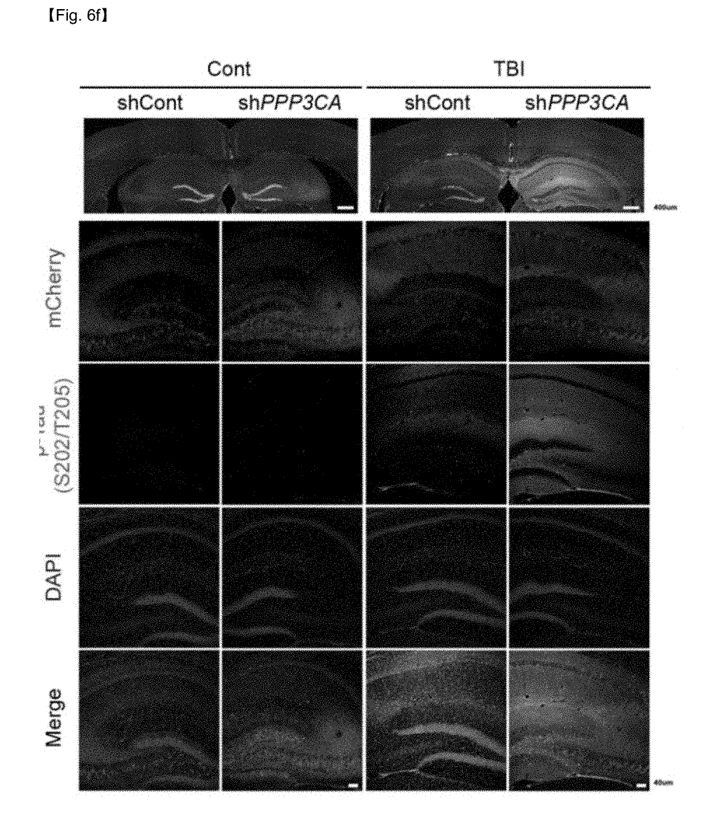

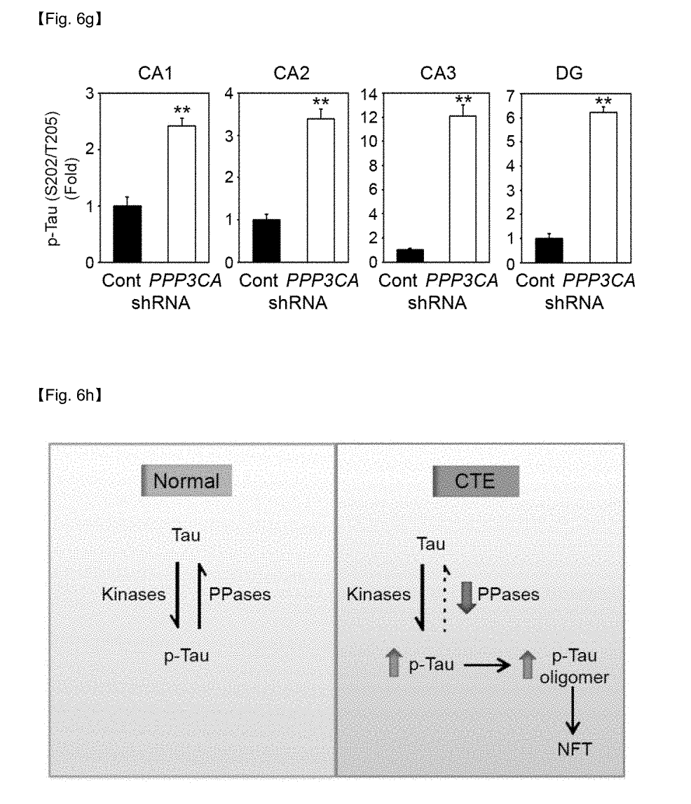

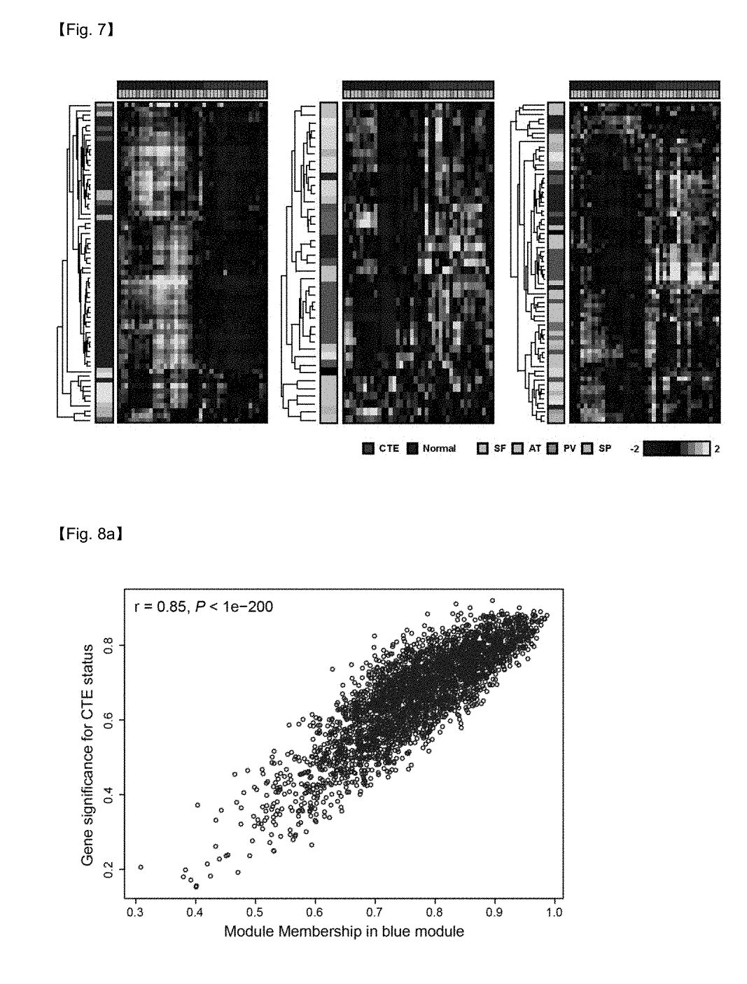

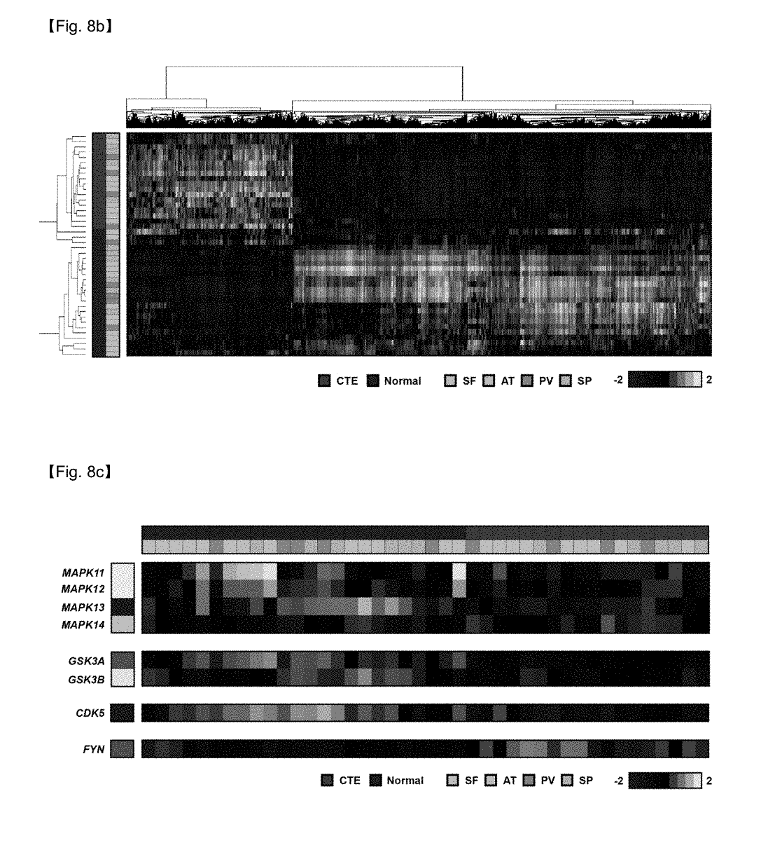

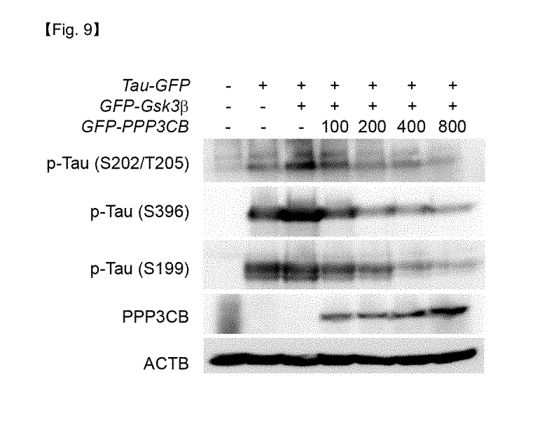

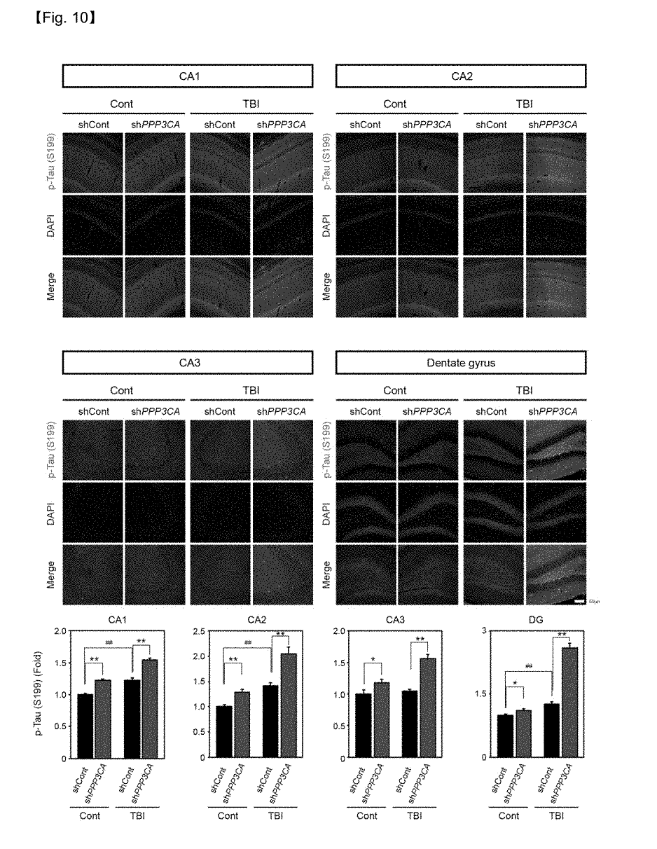

We conducted qPCR for PPP3CA in the AD subjects and confirmed downregulation, as shown in CTE subjects (FIG. 4a). We also observed a similar inverse correlation between the PPP3CA levels and p-tau in AD, as shown in CTE (FIGS. 4b and 4c). As we expected, not only the immunoreactivity of PPP3CA was decreased but tau protein also was hyperphosphorylated (S199, S396, S205/T208) in AD (FIGS. 4d and 4e). Confocal microscopy showed that the immunoreactivity of PPP3CA was reduced but that the immunoreactivity of p-tau was elevated in AD (FIG. 4f). Interestingly, we also observed the colocalization of PPP3CA with p-tau (S202/T205) in peripheral foci of neurofibrillary tangles in the cortex of AD.

### EXAMPLE 6

### Inhibition of PPP3CA/PP2B Activity Leads to Tau Phosphorylation and Tau Aggregation

Following the confirmation of decreased PPP3CA activity and elevated tau phosphorylation in human CTE and AD samples, we examined whether PPP3CA inhibition would be sufficient to increase tau phosphorylation and aggregation using in vitro cell line models of tauopathy. To this end, we used Tau-BiFC cell lines (SH-SY5Y and HEK293), which enable the conventional fluorescence imaging of tau phosphorylation and aggregation (FIG. 5a). Three different PP2A/PPP3CA inhibitors (okadaic acid, cyclosporine A, and deltamethrin) increased the level of tau phosphorylation in both SH-SY5Y and HEK293 Tau-BiFC cell lines (FIG. 5b). Okadaic acid and cyclosporine A treatment elevated the level of tau phosphorylation in a dose-dependent manner, whereas deltamethrin showed a declining trend after the threshold of 3 μM (FIG. 5c). Similar results were observed in the HEK 293 cell line, with a higher intensity of tau phosphorylation due to okadaic acid (FIG. 5d).

### EXAMPLE 7

### PPP3CA Directly Regulates Tau Phosphorylation

After confirming the involvement of PPP3CA catalytic activity in the dephosphorylation of tau, we further verified whether PPP3CA directly modulates tau phosphorylation (FIG. 6). Because GSK3□ is a well-known kinase of tau, we overexpressed GSK3□ with PPP3CA and tau and determined the level of phosphorylated tau. Whereas the overexpression of GSK3□ elevated tau phosphorylation at S202/T205 and S214, the cotransfection of PPP3CA reversed the effects of GSK3□□ and decreased the level of phosphorylated tau in a dose-dependent manner (FIG. 6a). In addition, PPP3CB, another differentially expressed subunit of PPP3CA, reversed the effects of GSK3□ and decreased the level of phosphorylated tau in a dose-dependent manner (FIG. 9). Regression analyses confirmed that the level of p-tau (S202/T205) was inversely correlated with the expression of PPP3CA (R=0.892) (FIG. 6b). Additionally, we examined whether the catalytic subunit of PPP3CA is necessary and sufficient to phosphorylate tau (FIG. 6c). To this end, we transfected cells with a catalytic site deletion mutant of PPP3CA (PPP3CAdeltaCAT) and verified that tau dephosphorylation did not occur with this mutant. The data showed that the catalytic activity of PPP3CA is indispensable for the dephosphorylation of p-tau. Moreover, we knocked down PPP3CA using shRNAs to demonstrate loss-of-function of PPP3CA (FIG. 6d), resulting in a marked increase of tau phosphorylation in response to GSK3β.

### EXAMPLE 8

### PPP3CA/PP2B Modulates Tau Dephosphorylation in In Vivo Animal Model of CTE

To verify whether the downregulation of PPP3CA affects the phosphorylation of tau in vivo, we transduced adeno-associated viral (AAV) vectors containing shRNA for PPP3CA into hippocampal regions of tau (P301L) transgenic mouse brains and measured the difference in tau phosphorylation compared with the control after multiple weight drop-induced head injuries (FIG. 6e). Consistent with the cellular model, the knockdown of PPP3CA using AAV-shPPP3CA markedly reduced the immunoreactivity of PPP3CA in the hippocampal regions of mice (FIG. 6f). Furthermore, the immunoreactivity of p-tau (S202/T205) was robustly increased in the dentate gyrus after exposure to weight drop-induced head injuries (FIG. 6g). The immunoreactivity of p-tau (S202/T205) was elevated in the region of PPP3CA knockdown using AAV-shPPP3CA, whereas p-tau (Ser202/Thr205) was not detected after AAV-shRNA control transduction (FIG. 6h). Similarly, the immunoreactivity of p-tau (S199) was enhanced in hippocampal regions (CA1, CA3, and DG) with PPP3CA knockdown using AAV-shPPP3CA upon weight drop-induced multiple head injuries (FIG. 10).

1. A method of prevention or treatment of neurodegenerative brain disease caused by traumatic brain injury, comprising administering to a subject a regulator increasing the expression or activity of calcineurin.

2. The method of claim 1, wherein the calcineurin is PPP3CA (catalytic subunit, alpha isozyme) or PP2B (Serine/threonine-protein phosphatase 2B).

3. The method according to claim 1, wherein the regulator increasing the activity of calcineurin is selected from the group consisting of compounds, peptides, peptide mimetics, aptamers, antibodies and natural products that are specifically binding to PPP3CA or PP2B.

4. The method of claim 1, wherein the regulator increasing the expression of calcineurin is an expression vector comprising nucleotide sequence encoding calcineurin or mRNAs.

5. The method of claim 1, wherein the neurodegenerative disease caused by traumatic brain injury is a neurodegenerative brain disease caused by aggregation or increased phosphorylation of Tau protein.

6. The method of claim 2, wherein the PPP3CA or PP2B inhibits phosphorylation or aggregation of Tau protein.

7. A method of screening a therapeutic agent for neurodegenerative brain disease caused by traumatic brain injury comprising:
treating neural tissue-derived cells with a candidate substance for a therapeutic agent;
measuring the expression or activity of calcineurin in the cells; and
determining the candidate substance as a therapeutic agent for neurodegenerative brain disease caused by traumatic brain injury, when the expression or activity of calcineurin in the cells treated by the candidate substance are increased more than in untreated cells.

8. The method of screening of claim 7, wherein the neural tissue-derived cells are nerve cells or glial cells.

9. The method of screening of claim 7, wherein the calcineurin is PPP3CA (protein phosphatase 3, catalytic subunit, alpha isozyme) or PP2B (Serine/threonine-protein phosphatase 2B).

10. The method of screening of claim 7, wherein the method for measuring the expression of calcineurin is carried out by at least one selected from the group consisting of Western blot, co-immunoprecipitation assay, ELISA (enzyme linked immunosorbent assay), real time PCR, electrophoresis, immunostaining, and FACS (Fluorescence activated cell sorter).

11. The method of screening of claim 7, wherein the neurodegenerative brain disease caused by traumatic brain injury is a neurodegenerative brain disease caused by aggregation or increase of phosphorylation of Tau protein.

12. A method of diagnosis of a neurodegenerative brain disease caused by traumatic brain injury, comprising administering an agent being capable of measuring inhibition of activity of calcineurin.

13. The method of claim 12, wherein the calcineurin is PPP3CA (protein phosphatase 3, catalytic subunit, alpha isozyme) or PP2B (Serine/threonine-protein phosphatase 2B).

14. The method of claim 12, wherein the agent is an antibody or an aptamer being capable of specifically binding to PPP3CA or PP2B.

15. The method of claim 12, wherein the neurodegenerative brain disease caused by traumatic brain injury is a neurodegenerative brain disease caused by aggregation or increase of Tau protein phosphorylation.

16. The method of claim 12, further comprising measuring inhibition of activity of PPP3CA or PP2B.

17. The method of claim 16, wherein the neurodegenerative brain disease caused by traumatic brain injury is determined, when the activity of PPP3CA or PP2B is inhibited.

18. A kit for diagnosis of a neurodegenerative brain disease caused by traumatic brain injury, comprising an agent being capable of measuring expression or activity of calcineurin.

19. The kit for diagnosis of claim 18, wherein the agent is a primer or a probe for a calcineurin gene, or antibody against calcineurin protein.

20. The kit for the diagnosis of claim 18, wherein the neurodegenerative brain disease is a neurodegenerative brain disease caused by aggregation or increase of Tau protein phosphorylation.
